SEQUENCE LISTING (118 sequences)

SEQ ID NO: 1 - protein, 12 aa, Artificial Sequence
  Polypeptides from African Swine Virus as vaccines for preventive and therapeutic use
YKDMVNIARSRG

SEQ ID NO: 2 - protein, 16 aa, Artificial Sequence
  Polypeptides from African Swine Virus as vaccines for preventive and therapeutic use
SRLTKSELEKKIKRSK

SEQ ID NO: 3 - protein, 10 aa, Artificial Sequence
  Polypeptides from African Swine Virus as vaccines for preventive and therapeutic use
TKLDQEEKKA

SEQ ID NO: 4 - protein, 23 aa, Artificial Sequence
  Polypeptides from African Swine Virus as vaccines for preventive and therapeutic use
RCAWEETKNIINDFLEIPEERCT

SEQ ID NO: 5 - protein, 11 aa, Artificial Sequence
  Polypeptides from African Swine Virus as vaccines for preventive and therapeutic use
WEETKNIINDF

SEQ ID NO: 6 - protein, 9 aa, Artificial Sequence
  Polypeptides from African Swine Virus as vaccines for preventive and therapeutic use
DFLEIPEER

SEQ ID NO: 7 - protein, 9 aa, Artificial Sequence
  Polypeptides from African Swine Virus as vaccines for preventive and therapeutic use
HEVPECREF

SEQ ID NO: 8 - protein, 7 aa, Artificial Sequence
  Polypeptides from African Swine Virus as vaccines for preventive and therapeutic use
TKETKNL

SEQ ID NO: 9 - protein, 10 aa, Artificial Sequence
  Polypeptides from African Swine Virus as vaccines for preventive and therapeutic use
IENMDDLQKG

SEQ ID NO: 10 - protein, 21 aa, Artificial Sequence
  Polypeptides from African Swine Virus as vaccines for preventive and therapeutic use
PRQQKKCSKAEECTCNNGSCS

SEQ ID NO: 11 - protein, 16 aa, Artificial Sequence
  Polypeptides from African Swine Virus as vaccines for preventive and therapeutic use
KCSKAEECTCNNGSCS

SEQ ID NO: 12 - protein, 9 aa, Artificial Sequence
  Polypeptides from African Swine Virus as vaccines for preventive and therapeutic use
KCSKAEECT

SEQ ID NO: 13 - protein, 9 aa, Artificial Sequence
  Polypeptides from African Swine Virus as vaccines for preventive and therapeutic use
LKEDSRDRT

SEQ ID NO: 14 - protein, 9 aa, Artificial Sequence
  Polypeptides from African Swine Virus as vaccines for preventive and therapeutic use
MEKIAEEDI

SEQ ID NO: 15 - protein, 7 aa, Artificial Sequence
  Polypeptides from African Swine Virus as vaccines for preventive and therapeutic use
LHDTREF

SEQ ID NO: 16 - protein, 14 aa, Artificial Sequence
  Polypeptides from African Swine Virus as vaccines for preventive and therapeutic use
PDKADNKPEDDEES

SEQ ID NO: 17 - protein, 17 aa, Artificial Sequence
  Polypeptides from African Swine Virus as vaccines for preventive and therapeutic use
NKPEDDEESGAKPKKKH

SEQ ID NO: 18 - protein, 12 aa, Artificial Sequence
  Polypeptides from African Swine Virus as vaccines for preventive and therapeutic use
AKPKKKHLFPKL

SEQ ID NO: 19 - protein, 8 aa, Artificial Sequence
  Polypeptides from African Swine Virus as vaccines for preventive and therapeutic use
TREGIKQS

SEQ ID NO: 20 - protein, 20 aa, Artificial Sequence
  Polypeptides from African Swine Virus as vaccines for preventive and therapeutic use
FRKRKNSTSLQSHIPSDEQL

SEQ ID NO: 21 - protein, 18 aa, Artificial Sequence
  Polypeptides from African Swine Virus as vaccines for preventive and therapeutic use
YKKQQPPKKVCKVDKDCG

SEQ ID NO: 22 - protein, 8 aa, Artificial Sequence
  Polypeptides from African Swine Virus as vaccines for preventive and therapeutic use
VCKDKDCG

SEQ ID NO: 23 - protein, 15 aa, Artificial Sequence
  Polypeptides from African Swine Virus as vaccines for preventive and therapeutic use
VCKVDKDCGSGEHCV

SEQ ID NO: 24 - protein, 19 aa, Artificial Sequence
  Polypeptides from African Swine Virus as vaccines for preventive and therapeutic use
DCGSGEHCVRGTCSTLSCL

SEQ ID NO: 25 - protein, 10 aa, Artificial Sequence
  Polypeptides from African Swine Virus as vaccines for preventive and therapeutic use
GSGEHCVRGT

SEQ ID NO: 26 - protein, 23 aa, Artificial Sequence
  Polypeptides from African Swine Virus as vaccines for preventive and therapeutic use
SCLDAVKMDKRNIKIDSKISSCE

SEQ ID NO: 27 - protein, 17 aa, Artificial Sequence
  Polypeptides from African Swine Virus as vaccines for preventive and therapeutic use
LDAVKMDKRNIKIDSKI

SEQ ID NO: 28 - protein, 10 aa, Artificial Sequence
  Polypeptides from African Swine Virus as vaccines for preventive and therapeutic use
LDAVKMDKRN

SEQ ID NO: 29 - protein, 12 aa, Artificial Sequence
  Polypeptides from African Swine Virus as vaccines for preventive and therapeutic use
MDKRNIKIDSKI

SEQ ID NO: 30 - protein, 13 aa, Artificial Sequence
  Polypeptides from African Swine Virus as vaccines for preventive and therapeutic use
ADEQQEFGKTRHP

SEQ ID NO: 31 - protein, 28 aa, Artificial Sequence
  Polypeptides from African Swine Virus as vaccines for preventive and therapeutic use
VCEKYCSWGTDDCTGWEYVGDEKEGTCY

SEQ ID NO: 32 - protein, 14 aa, Artificial Sequence
  Polypeptides from African Swine Virus as vaccines for preventive and therapeutic use
VCEKYCSWGTDDCT

SEQ ID NO: 33 - protein, 7 aa, Artificial Sequence
  Polypeptides from African Swine Virus as vaccines for preventive and therapeutic use
VCEKYCS

SEQ ID NO: 34 - protein, 18 aa, Artificial Sequence
  Polypeptides from African Swine Virus as vaccines for preventive and therapeutic use
DDCTGWEYVGDEKEGTCY

SEQ ID NO: 35 - protein, 10 aa, Artificial Sequence
  Polypeptides from African Swine Virus as vaccines for preventive and therapeutic use
WEYVGDEKEG

SEQ ID NO: 36 - protein, 16 aa, Artificial Sequence
  Polypeptides from African Swine Virus as vaccines for preventive and therapeutic use
KYGKDHIIALPRNHKH

SEQ ID NO: 37 - protein, 13 aa, Artificial Sequence
  Polypeptides from African Swine Virus as vaccines for preventive and therapeutic use
MKMEVIFKTDLRS

SEQ ID NO: 38 - protein, 9 aa, Artificial Sequence
  Polypeptides from African Swine Virus as vaccines for preventive and therapeutic use
VEIINSGRI

SEQ ID NO: 39 - protein, 8 aa, Artificial Sequence
  Polypeptides from African Swine Virus as vaccines for preventive and therapeutic use
VKYDIVKS

SEQ ID NO: 40 - protein, 13 aa, Artificial Sequence
  Polypeptides from African Swine Virus as vaccines for preventive and therapeutic use
GQGYTEHQAQEEW

SEQ ID NO: 41 - protein, 13 aa, Artificial Sequence
  Polypeptides from African Swine Virus as vaccines for preventive and therapeutic use
FEEETESSASSES

SEQ ID NO: 42 - protein, 12 aa, Artificial Sequence
  Polypeptides from African Swine Virus as vaccines for preventive and therapeutic use
HEKNDNETNECT

SEQ ID NO: 43 - protein, 15 aa, Artificial Sequence
  Polypeptides from African Swine Virus as vaccines for preventive and therapeutic use
FEQEPSSEEPKDSKL

SEQ ID NO: 44 - protein, 17 aa, Artificial Sequence
  Polypeptides from African Swine Virus as vaccines for preventive and therapeutic use
QKTVQHIEQYGKKAPDF

SEQ ID NO: 45 - protein, 13 aa, Artificial Sequence
  Polypeptides from African Swine Virus as vaccines for preventive and therapeutic use
GKKAPDFNKVIRA

SEQ ID NO: 46 - protein, 10 aa, Artificial Sequence
  Polypeptides from African Swine Virus as vaccines for preventive and therapeutic use
TPLKEEEKEV

SEQ ID NO: 47 - protein, 22 aa, Artificial Sequence
  Polypeptides from African Swine Virus as vaccines for preventive and therapeutic use
LKEEEKEVVRLMVIKLLKKNKL

SEQ ID NO: 48 - protein, 9 aa, Artificial Sequence
  Polypeptides from African Swine Virus as vaccines for preventive and therapeutic use
IKLLKKNKL

SEQ ID NO: 49 - protein, 6 aa, Artificial Sequence
  Polypeptides from African Swine Virus as vaccines for preventive and therapeutic use
PRHYGE

SEQ ID NO: 50 - protein, 15 aa, Artificial Sequence
  Polypeptides from African Swine Virus as vaccines for preventive and therapeutic use
SRKKKAAAAIEEEDI

SEQ ID NO: 51 - protein, 12 aa, Artificial Sequence
  Polypeptides from African Swine Virus as vaccines for preventive and therapeutic use
KKKAAAAAIEEE

SEQ ID NO: 52 - protein, 12 aa, Artificial Sequence
  Polypeptides from African Swine Virus as vaccines for preventive and therapeutic use
EVTPQPGTSKPA

SEQ ID NO: 53 - protein, 12 aa, Artificial Sequence
  Polypeptides from African Swine Virus as vaccines for preventive and therapeutic use
NRPATNKPVTDN

SEQ ID NO: 54 - protein, 13 aa, Artificial Sequence
  Polypeptides from African Swine Virus as vaccines for preventive and therapeutic use
NKPVTDNPVTDRL

SEQ ID NO: 55 - protein, 24 aa, Artificial Sequence
  Polypeptides from African Swine Virus as vaccines for preventive and therapeutic use
ASQTMSAIENLRQRNTYTHKDLEN

SEQ ID NO: 56 - protein, 17 aa, Artificial Sequence
  Polypeptides from African Swine Virus as vaccines for preventive and therapeutic use
IENLRQRNTYTHKDLEN

SEQ ID NO: 57 - protein, 15 aa, Artificial Sequence
  Polypeptides from African Swine Virus as vaccines for preventive and therapeutic use
ASQTMSAIENLRQRN

SEQ ID NO: 58 - protein, 9 aa, Artificial Sequence
  Polypeptides from African Swine Virus as vaccines for preventive and therapeutic use
ANDGKADKI

SEQ ID NO: 59 - protein, 12 aa, Artificial Sequence
  Polypeptides from African Swine Virus as vaccines for preventive and therapeutic use
NKSYGKPDPEPT

SEQ ID NO: 60 - protein, 10 aa, Artificial Sequence
  Polypeptides from African Swine Virus as vaccines for preventive and therapeutic use
GFEYNKVRPH

SEQ ID NO: 61 - protein, 11 aa, Artificial Sequence
  Polypeptides from African Swine Virus as vaccines for preventive and therapeutic use
FPRNGYDWDNQ

SEQ ID NO: 62 - protein, 38 aa, Artificial Sequence
  Polypeptides from African Swine Virus as vaccines for preventive and therapeutic use
YCEYPGERLYENVRFDVNGNSLDEYSSDVTTLVRKFCI

SEQ ID NO: 63 - protein, 24 aa, Artificial Sequence
  Polypeptides from African Swine Virus as vaccines for preventive and therapeutic use
GERLYENVRFDVNGNSLDEYSSDV

SEQ ID NO: 64 - protein, 12 aa, Artificial Sequence
  Polypeptides from African Swine Virus as vaccines for preventive and therapeutic use
GERLYENVRFDV

SEQ ID NO: 65 - protein, 15 aa, Artificial Sequence
  Polypeptides from African Swine Virus as vaccines for preventive and therapeutic use
GDKMTGYKHLVGQEV

SEQ ID NO: 66 - protein, 28 aa, Artificial Sequence
  Polypeptides from African Swine Virus as vaccines for preventive and therapeutic use
LCNIHDLHKPHQSKPILTDENDTQRTCS

SEQ ID NO: 67 - protein, 11 aa, Artificial Sequence
  Polypeptides from African Swine Virus as vaccines for preventive and therapeutic use
HDLHKPHQSKP

SEQ ID NO: 68 - protein, 9 aa, Artificial Sequence
  Polypeptides from African Swine Virus as vaccines for preventive and therapeutic use
TDENDTQRT

SEQ ID NO: 69 - protein, 9 aa, Artificial Sequence
  Polypeptides from African Swine Virus as vaccines for preventive and therapeutic use
IQTAGKQDI

SEQ ID NO: 70 - protein, 12 aa, Artificial Sequence
  Polypeptides from African Swine Virus as vaccines for preventive and therapeutic use
TDATYLDIRRNV

SEQ ID NO: 71 - protein, 11 aa, Artificial Sequence
  Polypeptides from African Swine Virus as vaccines for preventive and therapeutic use
IKLRFWFNENV

SEQ ID NO: 72 - protein, 19 aa, Artificial Sequence
  Polypeptides from African Swine Virus as vaccines for preventive and therapeutic use
GERFITIKLASQKDLVNEF

SEQ ID NO: 73 - protein, 32 aa, Artificial Sequence
  Polypeptides from African Swine Virus as vaccines for preventive and therapeutic use
QKDLVNEFPGLFIRQSRFIPGRPSRRNIRFKP

SEQ ID NO: 74 - protein, 12 aa, Artificial Sequence
  Polypeptides from African Swine Virus as vaccines for preventive and therapeutic use
GRPSRRNIRFKP

SEQ ID NO: 75 - protein, 14 aa, Artificial Sequence
  Polypeptides from African Swine Virus as vaccines for preventive and therapeutic use
TPEIHNLFVKRVRF

SEQ ID NO: 76 - protein, 11 aa, Artificial Sequence
  Polypeptides from African Swine Virus as vaccines for preventive and therapeutic use
TNNNNHHDEKL

SEQ ID NO: 77 - protein, 14 aa, Artificial Sequence
  Polypeptides from African Swine Virus as vaccines for preventive and therapeutic use
SDQNPHQHRDWHKF

SEQ ID NO: 78 - protein, 15 aa, Artificial Sequence
  Polypeptides from African Swine Virus as vaccines for preventive and therapeutic use
AEISFQDRDTALPDA

SEQ ID NO: 79 - protein, 42 aa, Artificial Sequence
  Polypeptides from African Swine Virus as vaccines for preventive and therapeutic use
ACSSISDISPVTYPITLPIIKNISVTAHGINLIDKFPSKFCS

SEQ ID NO: 80 - protein, 8 aa, Artificial Sequence
  Polypeptides from African Swine Virus as vaccines for preventive and therapeutic use
IDKFPSKF

SEQ ID NO: 81 - protein, 8 aa, Artificial Sequence
  Polypeptides from African Swine Virus as vaccines for preventive and therapeutic use
IKTPDDPG

SEQ ID NO: 82 - protein, 10 aa, Artificial Sequence
  Polypeptides from African Swine Virus as vaccines for preventive and therapeutic use
LKPREEYQPS

SEQ ID NO: 83 - protein, 14 aa, Artificial Sequence
  Polypeptides from African Swine Virus as vaccines for preventive and therapeutic use
SRAREFYISWDTDY

SEQ ID NO: 84 - protein, 16 aa, Artificial Sequence
  Polypeptides from African Swine Virus as vaccines for preventive and therapeutic use
SEDIRRGPGRPPKKRV

SEQ ID NO: 85 - protein, 13 aa, Artificial Sequence
  Polypeptides from African Swine Virus as vaccines for preventive and therapeutic use
PKKRVVPNFERKG

SEQ ID NO: 86 - protein, 13 aa, Artificial Sequence
  Polypeptides from African Swine Virus as vaccines for preventive and therapeutic use
FERKGILEKPVRP

SEQ ID NO: 87 - protein, 10 aa, Artificial Sequence
  Polypeptides from African Swine Virus as vaccines for preventive and therapeutic use
SRLEFSYDNP

SEQ ID NO: 88 - protein, 16 aa, Artificial Sequence
  Polypeptides from African Swine Virus as vaccines for preventive and therapeutic use
VRCTPTEITFFSRDQS

SEQ ID NO: 89 - protein, 9 aa, Artificial Sequence
  Polypeptides from African Swine Virus as vaccines for preventive and therapeutic use
IDGKNVNHY

SEQ ID NO: 90 - protein, 20 aa, Artificial Sequence
  Polypeptides from African Swine Virus as vaccines for preventive and therapeutic use
INRELVEKMFNSIDRSFLKI

SEQ ID NO: 91 - protein, 8 aa, Artificial Sequence
  Polypeptides from African Swine Virus as vaccines for preventive and therapeutic use
NRELVEKM

SEQ ID NO: 92 - protein, 8 aa, Artificial Sequence
  Polypeptides from African Swine Virus as vaccines for preventive and therapeutic use
IDRSFLKI

SEQ ID NO: 93 - protein, 22 aa, Artificial Sequence
  Polypeptides from African Swine Virus as vaccines for preventive and therapeutic use
HRYDKPETLFFIFTDFDIDKEC

SEQ ID NO: 94 - protein, 8 aa, Artificial Sequence
  Polypeptides from African Swine Virus as vaccines for preventive and therapeutic use
HRYDKPET

SEQ ID NO: 95 - protein, 9 aa, Artificial Sequence
  Polypeptides from African Swine Virus as vaccines for preventive and therapeutic use
TDFDIDKEC

SEQ ID NO: 96 - protein, 23 aa, Artificial Sequence
  Polypeptides from African Swine Virus as vaccines for preventive and therapeutic use
SEPELDMDLIEMEKSISEERLKN

SEQ ID NO: 97 - protein, 9 aa, Artificial Sequence
  Polypeptides from African Swine Virus as vaccines for preventive and therapeutic use
SEPELDMDL

SEQ ID NO: 98 - protein, 13 aa, Artificial Sequence
  Polypeptides from African Swine Virus as vaccines for preventive and therapeutic use
EMEKSISEERLKN

SEQ ID NO: 99 - protein, 29 aa, Artificial Sequence
  Polypeptides from African Swine Virus as vaccines for preventive and therapeutic use
IEMEKSISEERLKNYPLRWEFTSKQLKKT

SEQ ID NO: 100 - protein, 17 aa, Artificial Sequence
  Polypeptides from African Swine Virus as vaccines for preventive and therapeutic use
LRWEFTSKQLKKTFSDL

SEQ ID NO: 101 - protein, 13 aa, Artificial Sequence
  Polypeptides from African Swine Virus as vaccines for preventive and therapeutic use
TELVTIEKLGGDT

SEQ ID NO: 102 - protein, 12 aa, Artificial Sequence
  Polypeptides from African Swine Virus as vaccines for preventive and therapeutic use
SYHEMYKSSNKI

SEQ ID NO: 103 - protein, 22 aa, Artificial Sequence
  Polypeptides from African Swine Virus as vaccines for preventive and therapeutic use
TDKIRILCEENGNLIFQSEMDA

SEQ ID NO: 104 - protein, 11 aa, Artificial Sequence
  Polypeptides from African Swine Virus as vaccines for preventive and therapeutic use
VKIKQKELIDS

SEQ ID NO: 105 - protein, 9 aa, Artificial Sequence
  Polypeptides from African Swine Virus as vaccines for preventive and therapeutic use
FHEIIGSKG

SEQ ID NO: 106 - protein, 13 aa, Artificial Sequence
  Polypeptides from African Swine Virus as vaccines for preventive and therapeutic use
PKFKKIKQSVYEY

SEQ ID NO: 107 - protein, 15 aa, Artificial Sequence
  Polypeptides from African Swine Virus as vaccines for preventive and therapeutic use
FEEDKKMLELFVQKL

SEQ ID NO: 108 - protein, 13 aa, Artificial Sequence
  Polypeptides from African Swine Virus as vaccines for preventive and therapeutic use
FKYPEIEKYEVDG

SEQ ID NO: 109 - protein, 16 aa, Artificial Sequence
  Polypeptides from African Swine Virus as vaccines for preventive and therapeutic use
LEKFSQLYRSRINSEL

SEQ ID NO: 110 - protein, 28 aa, Artificial Sequence
  Polypeptides from African Swine Virus as vaccines for preventive and therapeutic use
SCAFLSKYNDYILKKDPYILTITPGLCF

SEQ ID NO: 111 - protein, 12 aa, Artificial Sequence
  Polypeptides from African Swine Virus as vaccines for preventive and therapeutic use
SKYNDYILKKDP

SEQ ID NO: 112 - protein, 22 aa, Artificial Sequence
  Polypeptides from African Swine Virus as vaccines for preventive and therapeutic use
FEDLNFKYLYNSDKNSQHDKDF

SEQ ID NO: 113 - protein, 11 aa, Artificial Sequence
  Polypeptides from African Swine Virus as vaccines for preventive and therapeutic use
SDKNSQHDKDF

SEQ ID NO: 114 - protein, 23 aa, Artificial Sequence
  Polypeptides from African Swine Virus as vaccines for preventive and therapeutic use
PDIDVEDLENIILSSVSQIKKQI

SEQ ID NO: 115 - protein, 17 aa, Artificial Sequence
  Polypeptides from African Swine Virus as vaccines for preventive and therapeutic use
IKKQIPRCKDAFNKIES

SEQ ID NO: 116 - protein, 19 aa, Artificial Sequence
  Polypeptides from African Swine Virus as vaccines for preventive and therapeutic use
MEQYIKDISQDSKNISPRI

SEQ ID NO: 117 - protein, 17 aa, Artificial Sequence
  Polypeptides from African Swine Virus as vaccines for preventive and therapeutic use
IKYYRDMIATKHQTMDP

SEQ ID NO: 118 - protein, 13 aa, Artificial Sequence
  Polypeptides from African Swine Virus as vaccines for preventive and therapeutic use
VKHVEKKLDMLDR

(19) United States
(12) Patent Application Publication
(10) Pub. No.: US 2008/0131449 A1
(43) Pub. Date: Jun. 5, 2008

(54) Polypeptides from African Swine Fever virus as vaccines for preventive and therapeutic use

(76) Inventors: Matthias Rath, Bocholtz (NL); Aleksandra Niedzwiecki, San Jose, CA (US)

Correspondence Address: INHOUSE IP, 280 COLORADO AVE, PALO ALTO, CA, 94301, US

(21) Appl. No.: 11/473,677

(22) Filed: Jun. 22, 2006

(51) Int. Cl. A61K 39/12; C07K 14/005

(52) U.S. Cl. 424/186.1; 530/324; 530/350

(57) ABSTRACT

The present invention generally relates to the use of selected polypeptides from African Swine Fever virus for the prevention and therapy of African Swine Fever infections as well as other infections, including immune deficiencies in mammals and humans.

### FIELD OF THE INVENTION

The present invention generally relates to the use of selected polypeptides from African Swine Fever virus for the prevention and therapy of African Swine Fever infections as well as other infections, including immune deficiencies in mammals and humans.

### BACKGROUND OF THE INVENTION

African Swine Fever is an endemic disease in sub-Saharan Africa and many other parts of the developing world. It is caused by the African Swine Fever virus that primarily replicates in macrophages and monocytes leading to the impairment of the structure and function of the immune system of the infected organisms. Until now the African Swine epidemic continues to spread despite all efforts to contain it. Thus, there is an objective need for effective, safe and affordable preventive and therapeutic approaches, in particular for effective vaccines, to control and eventually eradicate this disease.

Since the characteristic feature of the African Swine Fever virus is to impair the immune system and to cause immune deficiencies in its hosts the development of vaccines and other therapeutic approaches against the African Swine Fever virus has implications for other immune deficiencies or diseases. Several other viruses are also known to cause immunodeficiency-like syndromes in humans, including cytomegalovirus, Epstein Barr Virus, HIV and others. Moreover, a series of cases of so-called “idiopathic” immunodeficiencies have been documented that display CD4+ T-lymphocytopenia with opportunistic infections, but show no evidence of HIV infection (1).

Since antibodies for the African Swine Fever virus have been detected in humans, the possibility of human infection with the African Swine Fever virus exists and may thus far have escaped any systematic screening. Thus, any preventive and therapeutic approach to African Swine Fever can have far-reaching implications to control immune deficiency conditions in humans.

### SUMMARY OF THE INVENTION

Synthetic oligopeptides prepared from African Swine Fever virus proteins are effective in prevention, treatment and diagnosis of African Swine fever as well as for immune deficiencies in humans.

Oligopeptides are identified and selected by means of suitable algorithms from the known amino acid sequence of pathogenicity-mediating African Swine Fever virus proteins. Subsequently, these oligopeptides are tested in vitro for their ability to decrease or completely block infection by the African Swine Fever virus (prevention and therapy) or for their ability to raise antibodies to detect the virus (diagnosis). Ultimately, the successfully tested African Swine Fever virus oligopeptides can be used in veterinary and clinical medicine.

### BRIEF DESCRIPTION OF THE DRAWINGS

FIG. 1 shows the antigenicity scores derived from and according to the Hopp-Woods hydrophylicity algorithm for the protein p54, a 183 amino acid long structural protein of the African Swine Fever virus. Since p54 is involved in the pathogenesis of African Swine fever (2), interrupting this pathogenicity-mediating pathway will lead to a decrease or a complete block of infection by this virus.

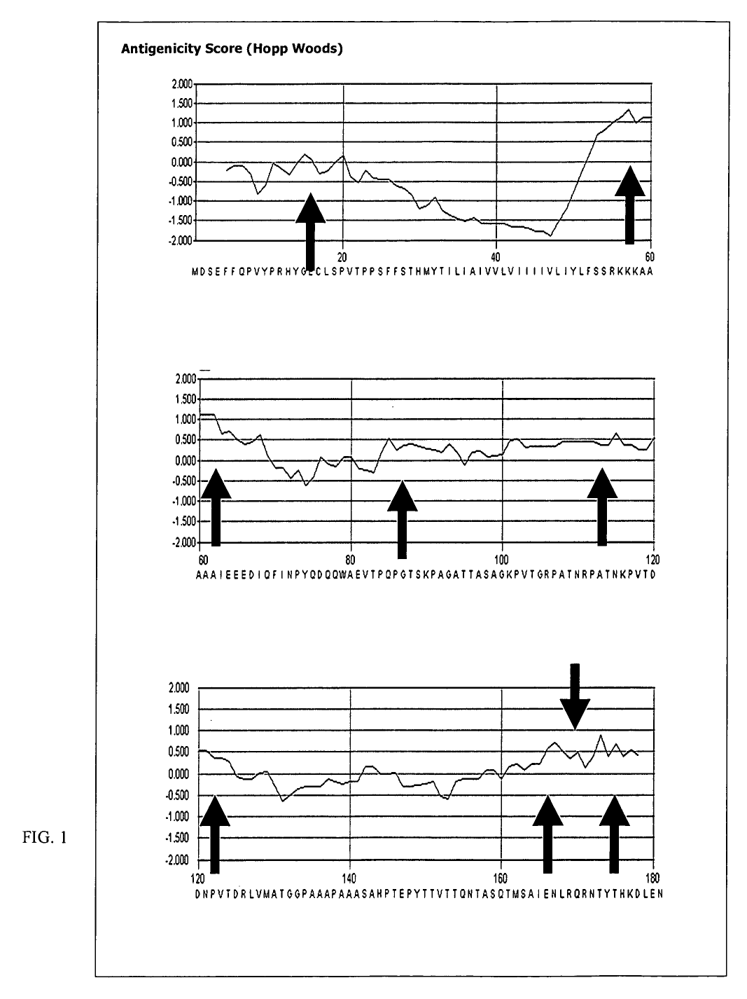

The relative peaks of this algorithm, defined as amino acid sequence regions of either high hydrophilic characteristics or sequence regions of higher hydrophilic characteristics in relation to adjacent amino acid sequences or in comparison to hydrophobic regions of the protein, represent the likely candidate sequence regions (oligopeptides) serving as epitopes (antigens) for antibody formation. Moreover these defined oligopeptide sequences represent the likely region by which a protein interacts with other proteins and/or biological compounds in an organism, including those interactions that mediate infection or other forms of pathogenicity.

Producing synthetic oligopeptides, corresponding to these algorithm maxima allows the development of preventive and therapeutic agents to control African Swine Fever virus infections. The relative peaks of the Hopp-Woods algorithm for the p54 protein of the African Swine Fever virus selected for this patent application are marked by arrows. The synthetic oligopeptides for the other African Swine Fever Virus proteins specified in this disclosure and the claims are selected in an analogous way.

### DETAILED DESCRIPTION OF THE INVENTION

The African Swine Fever virus is a particular virus the pathogenicity of which is largely determined by targeting the immune system of the host and disabling it.

Despite the DNA sequence of African Swine Fever virus having been determined (3), there is currently no effective vaccine available to control African Swine Fever as documented in the United Nations Food and Agricultural Organization's field handbook on this disease (4)

The present invention describes the identification and production of preventive and therapeutic agents, which—among others—can be used as vaccines against African Swine fever with the following specific steps being taken:
1. The identification of structural proteins and/or pathogenicity-mediating proteins and/or any other protein from the African Swine virus.
2. The analysis of the amino acid sequence of these proteins using specific algorithms allowing the determination of relative hydrophilic and/or polarity and/or charge and/or surface probability peaks and/or any other method allowing the determination of potential epitopes within these African Swine fever virus proteins.
3. The production of synthetic oligopeptides analogous to the epitope forming oligopeptides identified within the amino acid sequence of the African Swine fever virus proteins.
4. The modification of these synthetic oligopeptides to allow or improve antigencity and the formation of antibodies and/or to block pathogenicity of the African Swine fever virus in any other way by
   a. adding one or several predetermined amino acids to the selected oligopeptide sequence;
   b. subtracting one or several predetermined amino acids to the selected oligopeptide sequence;
   c. replacing one or several predetermined amino acids within the selected oligopeptide sequence;
   d. changing the linear topology of the selected oligopeptide to a cyclic topology;
   e. forming a linear chain of covalently bound repeats of the selected oligopeptide sequence;
   f. forming a cyclic chain of covalently bound repeats of the selected oligopeptide sequence;
   g. coupling an originally selected and/or modified oligopeptide to one or more haptens;
   h. to improve antigencity and enhance antibody formation in any other possible way;
   i. producing natural and/or synthetic peptidomimetics mimicking the three dimensional srtructure of the natural or modified oligopeptide.
5. To conduct in vitro and in vivo tests with the selected oligopeptides and/or peptidomimetics in order to establish their efficacy and efficiency as a therapeutic or diagnostic agents.
6. To identify those originally selected and/or modified synthetic oligopeptides and/or peptidomimetics for therapeutic or diagnostic use that
   a. display maximum and/or optimum ability to form antibodies against the African Swine Fever virus as potential therapeutic vaccines;
   b. display maximum and/or optimum competitive inhibition of pathogenicity mediating pathways of the African Swine Fever virus as potential therapeutic agents used for—but not limited to—acute therapeutic treatment of African Swine fever;
   c. display maximum and/or optimum antigenicity to raise antibodies for the development of tests to diagnose African Swine fever.
7. To use those originally selected and/or modified synthetic oligopeptides and/or peptidomimetics as therapeutic vaccines that display maximum and/or optimum ability to form antibodies against the African Swine virus.
8. To use those originally selected and/or modified synthetic oligopeptides and/or peptidomimetics as therapeutic vaccines that display maximum and/or optimum ability for competitive inhibition of pathogenicity mediating pathways of the African Swine Fever virus as potential therapeutic agents used for—but not limited to—the acute therapeutic treatment of African Swine fever.
9. To use those originally selected and/or modified synthetic oligopeptides and/or peptidomimetics that display maximum and/or optimum antigenicity to be used in the development of diagnostic tests or screening procedures for the African Swine virus.

The current invention also describes the application of the current invention for the diagnosis and treatment of immune deficiency conditions in mammals.

The following are the characteristics of the African Swine Virus:
1. it targets the immune system of the host
2. it has the following morphological features, in particular it structurally and functionally impairs the lymph nodes and other integral parts of the immune system;
3. its hematological changes includes a significant decrease of CD4 and T-cell counts;
4. it has clinical manifestations namely lymph node swelling, increased susceptibility to infections, and others;
5. it has both an acute and chronic form of infectious states;
6. it is known to display a high frequency of alteration of their genetic sequence in order to escape the host defense system;
7. it is endemic in sub-Saharan Africa and few other regions.

While the African Swine Fever virus has been primarily detected in pigs and certain other animals, antibodies against the African Swine Fever virus have also been found in humans (5). The fact that there was no description of any finding of the African Swine Fever virus in humans may thus be attributable to oversight or a lack of understanding for the significance of African Swine fever virus for the pathogenicity of immune deficiencies in humans.

Thus, the inventions described in this patent application can have far reaching implications not only for the control of African Swine fever but also for the control of other immunodeficiency diseases.

The main structural and/or pathogenicity mediating proteins of the African Swine virus are the following:

### Protein p10

Protein p10 of the African Swine Virus has the following relative hydrophilic peaks:

| | |
|---|---|
| Tyr-Lys-Asp-Met-VaI-Asn-Ile-Ala-Arg- | (SEQ ID NO: 1) |
| Ser-Arg-Gly-;; | |
| Ser-Arg-Leu-Thr-Lys-Ser-Glu-Leu-Glu- | (SEQ ID NO: 2) |
| Lys-Lys-Ile-Lys-Arg-Ser-Lys-. | |

### Protein p11.5

Protein p11.5 of the African Swine Virus has the following relative hydrophilic peaks:

| | |
|---|---|
| Thr-Lys-Leu-Asp-Gln-Glu-Glu-Lys-Lys- | (SEQ ID NO: 3) |
| Ala-; | |
| Arg-Cys-Ala-Trp-Glu-Glu-Thr-Lys-Asn- | (SEQ ID NO: 4) |
| Ile-Ile-Asn-Asp-Phe-Leu-Glu-Ile-Pro- | |
| Glu-Glu-Arg-Cys-Thr-; | |
| Trp-Glu-Glu-Thr-Lys-Asn-Ile-Ile-Asn- | (SEQ ID NO: 5) |
| Asp-Phe-; | |
| Asp-Phe-Leu-Glu-Ile-Pro-Glu-Glu- | (SEQ ID NO: 6) |
| Arg-; | |
| His-Glu-Val-Pro-Glu-Cys-Arg-Glu- | (SEQ ID NO: 7) |
| Phe-; | |
| Thr-Lys-Glu-Thr-Lys-Asn-Leu-; | (SEQ ID NO: 8) |
| Ile-Glu-Asn-Met-Asp-Asp-Leu-Gln-Lys- | (SEQ ID NO: 9) |
| Gly-. | |

### Protein p12

Protein p12 of the African Swine Virus has the following relative hydrophilic peaks:

| | |
|---|---|
| Pro-Arg-Gln-Gln-Lys-Lys-Cys-Ser- | (SEQ ID NO: 10) |
| Lys-Ala-Glu-Glu-Cys-Thr-Cys-Asn- | |
| Asn-Gly-Ser-Cys-Ser-; | |
| Lys-Cys-Ser-Lys-Ala-Glu-Glu-Cys- | (SEQ ID NO: 11) |
| Thr-Cys-Asn-Asn-Gly-Ser-Cys-Ser-; | |
| Lys-Cys-Ser-Lys-Ala-Glu-Glu-Cys- | (SEQ ID NO: 12) |
| Thr-. | |

### Protein p14.5

Protein p14.5 of the African Swine Virus has the following relative hydrophilic peaks:

| | |
|---|---|
| Leu-Lys-Glu-Asp-Ser-Arg-Asp-Arg- | (SEQ ID NO: 13) |
| Thr-; | |
| Met-Glu-Lys-Ile-Ala-Glu-Glu-Asp- | (SEQ ID NO: 14) |
| Ile-; | |
| Leu-His-Asp-Thr-Arg-Glu-Phe-; | (SEQ ID NO: 15) |
| Pro-Asp-Lys-Ala-Asp-Asn-Lys-Pro- | (SEQ ID NO: 16) |
| Glu-Asp-Asp-Glu-Glu-Ser-; | |
| Asn-Lys-Pro-Glu-Asp-Asp-Glu-Glu- | (SEQ ID NO: 17) |
| Ser-Gly-Ala-Lys-Pro-Lys-Lys-Lys- | |
| His-; | |
| Ala-Lys-Pro-Lys-Lys-Lys-His-Leu- | (SEQ ID NO: 18) |
| Phe-Pro-Lys-Leu-. | |

### Protein p17

Protein p17 of the African Swine Virus has the following relative hydrophilic peaks:

| | |
|---|---|
| Thr-Arg-Glu-Gly-Ile-Lys-Gln-Ser-; | (SEQ ID NO: 19) |
| Phe-Arg-Lys-Arg-Lys-Asn-Ser-Thr- | (SEQ ID NO: 20) |
| Ser-Leu-Gln-Ser-His-Ile-Pro-Ser- | |
| Asp-Glu-Gln-Leu-. | |

### Protein p22

Protein p22 of the African Swine Virus has the following relative hydrophilic peaks:

| | |
|---|---|
| Tyr-Lys-Lys-Gln-Gln-Pro-Pro-Lys- | (SEQ ID NO: 21) |
| Lys-Val-Cys-Lys-Val-Asp-Lys-Asp- | |
| Cys-Gly-; | |
| Val-Cys-Lys-Asp-Lys-Asp-Cys-Gly-; | (SEQ ID NO: 22) |
| Val-Cys-Lys-Val-Asp-Lys-Asp-Cys- | (SEQ ID NO: 23) |
| Gly-Ser-Gly-Glu-His-Cys-Val-; | |
| Asp-Cys-Gly-Ser-Gly-Glu-His-Cys- | (SEQ ID NO: 24) |
| Val-Arg-Gly-Thr-Cys-Ser-Thr-Leu- | |
| Ser-Cys-Leu-; | |
| Gly-Ser-Gly-Glu-His-Cys-Val-Arg- | (SEQ ID NO: 25) |
| Gly-Thr-; | |
| Ser-Cys-Leu-Asp-Ala-Val-Lys-Met- | (SEQ ID NO: 26) |
| Asp-Lys-Arg-Asn-Ile-Lys-Ile-Asp- | |
| Ser-Lys-Ile-Ser-Ser-Cys-Glu-; | |
| Leu-Asp-Ala-Val-Lys-Met-Asp-Lys- | (SEQ ID NO: 27) |
| Arg-Asn-Ile-Lys-Ile-Asp-Ser-Lys- | |
| Ile- | |
| Leu-Asp-Ala-Val-Lys-Met-Asp-Lys- | (SEQ ID NO: 28) |
| Arg-Asn-; | |
| Met-Asp-Lys-Arg-Asn-Ile-Lys-Ile- | (SEQ ID NO: 29) |
| Asp-Ser-Lys-Ile-; | |
| Ala-Asp-Glu-Gln-Gln-Glu-Phe-Gly- | (SEQ ID NO: 30) |
| Lys-Thr-Arg-His-Pro-; | |
| Val-Cys-Glu-Lys-Tyr-Cys-Ser-Trp- | (SEQ ID NO: 31) |
| Gly-Thr-Asp-Asp-Cys-Thr-Gly-Trp- | |
| Glu-Tyr-Val-Gly-Asp-Glu-Lys-Glu- | |
| Gly-Thr-Cys-Tyr-; | |
| Val-Cys-Glu-Lys-Tyr-Cys-Ser-Trp- | (SEQ ID NO: 32) |
| Gly-Thr-Asp-Asp-Cys-Thr-; | |
| Val-Cys-Glu-Lys-Tyr-Cys-Ser-; | (SEQ ID NO: 33) |
| Asp-Asp-Cys-Thr-Gly-Trp-Glu-Tyr- | (SEQ ID NO: 34) |
| Val-Gly-Asp-Glu-Lys-Glu-Gly-Thr- | |
| Cys-Tyr-; | |
| Trp-Glu-Tyr-Val-Gly-Asp-Glu-Lys- | (SEQ ID NO: 35) |
| Glu-Gly-; | |
| Lys-Tyr-Gly-Lys-Asp-His-Ile-Ile- | (SEQ ID NO: 36) |
| Ala-Leu-Pro-Arg-Asn-His-Lys-His-. | |

### Protein p30

Protein p30 of the African Swine Virus has the following relative hydrophilic peaks:

| | |
|---|---|
| Met-Lys-Met-Glu-Val-Ile-Phe-Lys- | (SEQ ID NO: 37) |
| Thr-Asp-Leu-Arg-Ser-; | |
| Val-Glu-Ile-Ile-Asn-Ser-Gly-Arg- | (SEQ ID NO: 38) |
| Ile-; | |
| Val-Lys-Tyr-Asp-Ile-Val-Lys-Ser-; | (SEQ ID NO: 39) |
| Gly-Gln-Gly-Tyr-Thr-Glu-His-Gln- | (SEQ ID NO: 40) |
| Ala-Gln-Glu-Glu-Trp-; | |
| Phe-Glu-Glu-Glu-Thr-Glu-Ser-Ser- | (SEQ ID NO: 41) |
| Ala-Ser-Ser-Glu-Ser-; | |
| His-Glu-Lys-Asn-Asp-Asn-Glu-Thr- | (SEQ ID NO: 42) |
| Asn-Glu-Cys-Thr-; | |
| Phe-Glu-Gln-Glu-Pro-Ser-Ser-Glu- | (SEQ ID NO: 43) |
| Glu-Pro-Lys-Asp-Ser-Lys-Leu-; | |
| Gln-Lys-Thr-Val-Gln-His-Ile-Glu- | (SEQ ID NO: 44) |
| Gln-Tyr-Gly-Lys-Lys-Ala-Pro-Asp- | |
| Phe-; | |
| Gly-Lys-Lys-Ala-Pro-Asp-Phe-Asn- | (SEQ ID NO: 45) |
| Lys-Val-Ile-Arg-Ala-; | |
| Thr-Pro-Leu-Lys-Glu-Glu-Glu-Lys- | (SEQ ID NO: 46) |
| Glu-Val-; | |
| Leu-Lys-Glu-Glu-Glu-Lys-Glu-Val- | (SEQ ID NO: 47) |
| Val-Arg-Leu-Met-Val-Ile-Lys-Leu- | |
| Leu-Lys-Lys-Asn-Lys-Leu-; | |
| Ile-Lys-Leu-Leu-Lys-Lys-Asn-Lys- | (SEQ ID NO: 48) |
| Leu-. | |

### Protein p54

Protein p54 of the African Swine Virus has the following relative hydrophilic peaks:

| | |
|---|---|
| Pro-Arg-His-Tyr-Gly-Glu-; | (SEQ ID NO: 49) |
| Ser-Arg-Lys-Lys-Lys-Ala-Ala-Ala- | (SEQ ID NO: 50) |
| Ala-Ile-Glu-Glu-Glu-Asp-Ile-; | |
| Lys-Lys-Lys-Ala-Ala-Ala-Ala-Ala- | (SEQ ID NO: 51) |
| Ile-Glu-Glu-Glu-; | |
| Glu-Val-Thr-Pro-Gln-Pro-Gly-Thr- | (SEQ ID NO: 52) |
| Ser-Lys-Pro-Ala-; | |
| Asn-Arg-Pro-Ala-Thr-Asn-Lys-Pro- | (SEQ ID NO: 53) |
| Val-Thr-Asp-Asn-; | |
| Asn-Lys-Pro-Val-Thr-Asp-Asn-Pro- | (SEQ ID NO: 54) |
| Val-Thr-Asp-Arg-Leu-; | |
| Ala-Ser-Gln-Thr-Met-Ser-Ala-Ile- | (SEQ ID NO: 55) |
| Glu-Asn-Leu-Arg-Gln-Arg-Asn-Thr- | |
| Tyr-Thr-His-Lys-Asp-Leu-Glu-Asn-; | |
| Ile-Glu-Asn-Leu-Arg-Gln-Arg-Asn- | (SEQ ID NO: 56) |
| Thr-Tyr-Thr-His-Lys-Asp-Leu-Glu- | |
| Asn-; | |
| Ala-Ser-Gln-Thr-Met-Ser-Ala-Ile- | (SEQ ID NO: 57) |
| Glu-Asn-Leu-Arg-Gln-Arg-Asn-. | |

### Protein p72

Protein p72 of the African Swine Virus has the following relative hydrophilic peaks:

| | |
|---|---|
| Ala-Asn-Asp-Gly-Lys-Ala-Asp-Lys- | (SEQ ID NO: 58) |
| Ile-; | |
| Asn-Lys-Ser-Tyr-Gly-Lys-Pro-Asp- | (SEQ ID NO: 59) |
| Pro-Glu-Pro-Thr-; | |
| Gly-Phe-Glu-Tyr-Asn-Lys-Val-Arg- | (SEQ ID NO: 60) |
| Pro-His-; | |
| Phe-Pro-Arg-Asn-Gly-Tyr-Asp-Trp- | (SEQ ID NO: 61) |
| Asp-Asn-Gln-; | |
| Tyr-Cys-Glu-Tyr-Pro-Gly-Glu-Arg- | (SEQ ID NO: 62) |
| Leu-Tyr-Glu-Asn-Val-Arg-Phe-Asp- | |
| Val-Asn-Gly-Asn-Ser-Leu-Asp-Glu- | |
| Tyr-Ser-Ser-Asp-Val-Thr-Thr-Leu- | |
| Val-Arg-Lys-Phe-Cys-Ile-; | |
| Gly-Glu-Arg-Leu-Tyr-Glu-Asn-Val- | (SEQ ID NO: 63) |
| Arg-Phe-Asp-Val-Asn-Gly-Asn-Ser- | |
| Leu-Asp-Glu-Tyr-Ser-Ser-Asp-Val-; | |
| Gly-Glu-Arg-Leu-Tyr-Glu-Asn-Val- | (SEQ ID NO: 64) |
| Arg-Phe-Asp-Val-; | |
| Gly-Asp-Lys-Met-Thr-Gly-Tyr-Lys- | (SEQ ID NO: 65) |
| His-Leu-Val-Gly-Gln-Glu-Val-; | |
| Leu-Cys-Asn-Ile-His-Asp-Leu-His- | (SEQ ID NO: 66) |
| Lys-Pro-His-Gln-Ser-Lys-Pro-Ile- | |
| Leu-Thr-Asp-Glu-Asn-Asp-Thr-Gln- | |
| Arg-Thr-Cys-Ser-; | |
| His-Asp-Leu-His-Lys-Pro-His-Gln- | (SEQ ID NO: 67) |
| Ser-Lys-Pro-; | |
| Thr-Asp-Glu-Asn-Asp-Thr-Gln-Arg- | (SEQ ID NO: 68) |
| Thr-; | |
| Ile-Gln-Thr-Ala-Gly-Lys-Gln-Asp- | (SEQ ID NO: 69) |
| Ile-; | |
| Thr-Asp-Ala-Thr-Tyr-Leu-Asp-Ile- | (SEQ ID NO: 70) |
| Arg-Arg-Asn-Val-; | |
| Ile-Lys-Leu-Arg-Phe-Trp-Phe-Asn- | (SEQ ID NO: 71) |
| Glu-Asn-Val-; | |
| Gly-Glu-Arg-Phe-Ile-Thr-Ile-Lys- | (SEQ ID NO: 72) |
| Leu-Ala-Ser-Gln-Lys-Asp-Leu-Val- | |
| Asn-Glu-Phe-; | |
| Gln-Lys-Asp-Leu-Val-Asn-Glu-Phe- | (SEQ ID NO: 73) |
| Pro-Gly-Leu-Phe-Ile-Arg-Gln-Ser- | |
| Arg-Phe-Ile-Pro-Gly-Arg-Pro-Ser- | |
| Arg-Arg-Asn-Ile-Arg-Phe-Lys-Pro-; | |
| Gly-Arg-Pro-Ser-Arg-Arg-Asn-Ile- | (SEQ ID NO: 74) |
| Arg-Phe-Lys-Pro-; | |
| Thr-Pro-Glu-Ile-His-Asn-Leu-Phe- | (SEQ ID NO: 75) |
| Val-Lys-Arg-Val-Arg-Phe-; | |
| Thr-Asn-Asn-Asn-Asn-His-His-Asp- | (SEQ ID NO: 76) |
| Glu-Lys-Leu-; | |
| Ser-Asp-Gln-Asn-Pro-His-Gln-His- | (SEQ ID NO: 77) |
| Arg-Asp-Trp-His-Lys-Phe-; | |
| Ala-Glu-Ile-Ser-Phe-Gln-Asp-Arg- | (SEQ ID NO: 78) |
| Asp-Thr-Ala-Leu-Pro-Asp-Ala-; | |
| Ala-Cys-Ser-Ser-Ile-Ser-Asp-Ile- | (SEQ ID NO: 79) |
| Ser-Pro-Val-Thr-Tyr-Pro-Ile-Thr- | |
| Leu-Pro-Ile-Ile-Lys-Asn-Ile-Ser- | |
| Val-Thr-Ala-His-Gly-Ile-Asn-Leu- | |
| Ile-Asp-Lys-Phe-Pro-Ser-Lys-Phe- | |
| Cys-Ser-; | |
| Ile-Asp-Lys-Phe-Pro-Ser-Lys-Phe-; | (SEQ ID NO: 80) |
| Ile-Lys-Thr-Pro-Asp-Asp-Pro-Gly-; | (SEQ ID NO: 81) |
| Leu-Lys-Pro-Arg-Glu-Glu-Tyr-Gln- | (SEQ ID NO: 82) |
| Pro-Ser-; | |
| Ser-Arg-Ala-Arg-Glu-Phe-Tyr-Ile- | (SEQ ID NO: 83) |
| Ser-Trp-Asp-Thr-Asp-Tyr-. | |

Proliferating Antigen-Like Proteins with Relative Hydrophilic Peaks

| | |
|---|---|
| Ser-Glu-Asp-Ile-Arg-Arg-Gly-Pro- | (SEQ ID NO: 74) |
| Gly-Arg-Pro-Pro-Lys-Lys-Arg-Val-; | |
| Pro-Lys-Lys-Arg-Val-Val-Pro-Asn- | (SEQ ID NO: 85) |
| Phe-Glu-Arg-Lys-Gly-; | |
| Phe-Glu-Arg-Lys-Gly-Ile-Leu-Glu- | (SEQ ID NO: 86) |
| Lys-Pro-Val-Arg-Pro-; | |
| Ser-Arg-Leu-Glu-Phe-Ser-Tyr-Asp- | (SEQ ID NO: 87) |
| Asn-Pro-; | |
| Val-Arg-Cys-Thr-Pro-Thr-Glu-Ile- | (SEQ ID NO: 88) |
| Thr-Phe-Phe-Ser-Arg-Asp-Gln-Ser-; | |
| Ile-Asp-Gly-Lys-Asn-Val-Asn-His- | (SEQ ID NO: 89) |
| Tyr-; | |
| Ile-Asn-Arg-Glu-Leu-Val-Glu-Lys- | (SEQ ID NO: 90) |
| Met-Phe-Asn-Ser-Ile-Asp-Arg-Ser- | |
| Phe-Leu-Lys-Ile-; | |
| Asn-Arg-Glu-Leu-Val-Glu-Lys-Met-; | (SEQ ID NO: 91) |
| Ile-Asp-Arg-Ser-Phe-Leu-Lys-Ile-; | (SEQ ID NO: 92) |
| His-Arg-Tyr-Asp-Lys-Pro-Glu-Thr- | (SEQ ID NO: 93) |
| Leu-Phe-Phe-Ile-Phe-Thr-Asp-Phe- | |
| Asp-Ile-Asp-Lys-Glu-Cys-; | |
| His-Arg-Tyr-Asp-Lys-Pro-Glu-Thr-; | (SEQ ID NO: 94) |
| Thr-Asp-Phe-Asp-Ile-Asp-Lys-Glu- | (SEQ ID NO: 95) |
| Cys-; | |
| Ser-Glu-Pro-Glu-Leu-Asp-Met-Asp- | (SEQ ID NO: 96) |
| Leu-Ile-Glu-Met-Glu-Lys-Ser-Ile- | |
| Ser-Glu-Glu-Arg-Leu-Lys-Asn-; | |
| Ser-Glu-Pro-Glu-Leu-Asp-Met-Asp- | (SEQ ID NO: 97) |
| Leu-; | |
| Glu-Met-Glu-Lys-Ser-Ile-Ser-Glu- | (SEQ ID NO: 98) |
| Glu-Arg-Leu-Lys-Asn-; | |
| Ile-Glu-Met-Glu-Lys-Ser-Ile-Ser- | (SEQ ID NO: 99) |
| Glu-Glu-Arg-Leu-Lys-Asn-Tyr-Pro- | |
| Leu-Arg-Trp-Glu-Phe-Thr-Ser-Lys- | |
| Gln-Leu-Lys-Lys-Thr-; | |
| Leu-Arg-Trp-Glu-Phe-Thr-Ser-Lys- | (SEQ ID NO: 100) |
| Gln-Leu-Lys-Lys-Thr-Phe-Ser-Asp- | |
| Leu-; | |
| Thr-Glu-Leu-Val-Thr-Ile-Glu-Lys- | (SEQ ID NO: 101) |
| Leu-Gly-Gly-Asp-Thr-; | |
| Ser-Tyr-His-Glu-Met-Tyr-Lys-Ser- | (SEQ ID NO: 102) |
| Ser-Asn-Lys-Ile-; | |
| Thr-Asp-Lys-Ile-Arg-Ile-Leu-Cys- | (SEQ ID NO: 103) |
| Glu-Glu-Asn-Gly-Asn-Leu-Ile-Phe- | |
| Gln-Ser-Glu-Met-Asp-Ala-. | |

### Serine Proteinase Inhibitor Relative Hydrophilic Peaks

| | |
|---|---|
| Val-Lys-Ile-Lys-Gln-Lys-Glu-Leu- | (SEQ ID NO: 104) |
| Ile-Asp-Ser-; | |
| Phe-His-Glu-Ile-Ile-Gly-Ser-Lys- | (SEQ ID NO: 105) |
| Gly-; | |
| Pro-Lys-Phe-Lys-Lys-Ile-Lys-Gln- | (SEQ ID NO: 106) |
| Ser-Val-Tyr-Glu-Tyr-; | |
| Phe-Glu-Glu-Asp-Lys-Lys-Met-Leu- | (SEQ ID NO: 107) |
| Glu-Leu-Phe-Val-Gln-Lys-Leu-; | |
| Phe-Lys-Tyr-Pro-Glu-Ile-Glu-Lys- | (SEQ ID NO: 108) |
| Tyr-Glu-Val-Asp-Gly-; | |
| Leu-Glu-Lys-Phe-Ser-Gln-Leu-Tyr- | (SEQ ID NO: 109) |
| Arg-Ser-Arg-Ile-Asn-Ser-Glu-Leu-; | |
| Ser-Cys-Ala-Phe-Leu-Ser-Lys-Tyr- | (SEQ ID NO: 110) |
| Asn-Asp-Tyr-Ile-Leu-Lys-Lys-Asp- | |
| Pro-Tyr-Ile-Leu-Thr-Ile-Thr-Pro- | |
| Gly-Leu-Cys-Phe-; | |
| Ser-Lys-Tyr-Asn-Asp-Tyr-Ile-Leu- | (SEQ ID NO: 111) |
| Lys-Lys-Asp-Pro-; | |
| Phe-Glu-Asp-Leu-Asn-Phe-Lys-Tyr- | (SEQ ID NO: 112) |
| Leu-Tyr-Asn-Ser-Asp-Lys-Asn-Ser- | |
| Gln-His-Asp-Lys-Asp-Phe-; | |
| Ser-Asp-Lys-Asn-Ser-Gln-His-Asp- | (SEQ ID NO: 113) |
| Lys-Asp-Phe-; | |
| Pro-Asp-Ile-Asp-Val-Glu-Asp-Leu- | (SEQ ID NO: 114) |
| Glu-Asn-Ile-Ile-Leu-Ser-Ser-Val- | |
| Ser-Gln-Ile-Lys-Lys-Gln-Ile-; | |
| Ile-Lys-Lys-Gln-Ile-Pro-Arg-Cys- | (SEQ ID NO: 115) |
| Lys-Asp-Ala-Phe-Asn-Lys-Ile-Glu- | |
| Ser-; | |
| Met-Glu-Gln-Tyr-Ile-Lys-Asp-Ile- | (SEQ ID NO: 116) |
| Ser-Gln-Asp-Ser-Lys-Asn-Ile-Ser- | |
| Pro-Arg-Ile-; | |
| Ile-Lys-Tyr-Tyr-Arg-Asp-Met-Ile- | (SEQ ID NO: 117) |
| Ala-Thr-Lys-His-Gln-Thr-Met-Asp- | |
| Pro-; | |
| Val-Lys-His-Val-Glu-Lys-Lys-Leu- | (SEQ ID NO: 118) |
| Asp-Met-Leu-Asp-Arg. | |

### REFERENCES CITED

1. Duncan R A et al. Idiopathic CD4+ T-lymphocytopenia—four patients with opportunistic infections and no evidence of HIV infection. N Engl J Med. 1993; 328(6): 393-8.

2. Rodriguez F et al. Characterization and molecular basis of heterogeneity of the African Swine Fever virus envelope protein p54. J Virol 1994; 68 (11): 7244-7252.

3. Yanez R J et al. Analysis of the complete nucleotide sequence of African Swine Fever virus. Virology 1995; 208: 249-278.

4. Recognizing African Swine Fever, A Field Manual, UN Food and Agricultural Organization, ISBN 92-5-104471-6

5. Hess W R. African Swine Fever: A Reassessment. Advances in Veterinary Science and Comparative Medicine Vol. 25. Cornelius C E, Simpson C F eds. Academic Press, New York, 1981:39-69.

What is claimed is:

1. Oligopeptides selected from the group consisting of SEQ ID NO: 1 through SEQ ID NO: 118.

2. Modification of any of the sequences identified as SEQ ID No: 1 through SEQ ID NO: 118 by omitting one or several predetermined amino residues at the N-terminal end.

3. Modification of any of the sequences identified as SEQ ID No: 1 through SEQ ID NO: 118 by omitting one or several predetermined amino acid residues at the C-terminal end.

4. Modification of any of the sequences identified as SEQ ID No: 1 through SEQ ID NO: 118 by omitting one or several predetermined amino acid residues at the N-terminal and the C-terminal end.

5. Modification of any of the sequences identified as SEQ ID No: 1 through SEQ ID NO: 118 by substituting one or several predetermined amino acid residues within the given sequence without consideration of charge and polarity of the substitution residue.

6. Modification of any of the sequences identified as SEQ ID No: 1 through SEQ ID NO: 118 by substituting one or several predetermined of the amino acid residues within the given sequence with amino acid residues with similar charge and/or polarity.

7. Modification of any of the sequences identified as SEQ ID No: 1 through SEQ ID NO: 118 by omitting one or several predetermined amino acid residues within the given sequence.

8. Modification of any of the sequences identified as SEQ ID No: 1 through SEQ ID NO: 118 by repeating the oligopeptides sequence one or more times each of them covalently bound to one or several predetermined oligopeptides repeat(s) with linear topology or other peptidomemetic.

9. Modification of any of the sequences identified as SEQ ID No: 1 through SEQ ID NO: 118 by using cyclic oligopeptide topology instead of linear oligopeptide topology or other peptidomemetic.

10. Modification of any of the sequences listed identified as SEQ ID No: 1 through SEQ ID NO: 118 by repeating the oligopeptide sequence one or more times each of them covalently bound to one or more oligopeptides repeat(s) with cyclic topology or other cyclic peptidomemetic.

11. Modification of those sequences identified as SEQ ID No: 1 through SEQ ID NO: 118 that contain two residues of the amino acid cysteine to form disulfide bonds thereby changing the secondary and tertiary structure of the oligopeptide as well as epitope formation.

12. Modification of any of the sequences identified as SEQ ID No: 1 through SEQ ID NO: 118 by a combination of two or more of the modifications of claims 2 to 10.

13. The production of natural and/or synthetic peptidomimetics mimicking the three dimensional structure of an oligopeptide sequence according to claim 1 to 10 and/or mimicking the three dimensional structure of a modification of such an oligopeptide according to claims 2 to 10.

14. The preventive or therapeutic use of one or more of the oligopeptides identified as SEQ ID No: 1 through SEQ ID NO: 118 and/or modifications thereof according to claims 2 to 10 and/or peptidomimetics according to claim 1 to 10 to directly and competitively reduce or block infections by the African Swine Fever virus.

15. The preventive or therapeutic use of one or more of the oligopeptides identified as SEQ ID No: 1 through SEQ ID NO: 118 and/or modifications thereof according to claims 2 to 10 and/or peptidomimetics according to claim 1 to 10 to indirectly reduce or block the metabolic action or interaction of African Swine Fever virus by applying them as vaccines by subcutaneous application or in another acceptable way to stimulate a specific immune response which can partially or completely block infections by the African Swine Fever virus.

16. The preventive or therapeutic use of one or more of the oligopeptides identified as SEQ ID No: 1 through SEQ ID NO: 118 and/or modifications thereof according to claims 2 to 10 and/or peptidomimetics according to claim 1 to 10 to directly and competitively reduce or block immune deficiencies.

17. The preventive or therapeutic use of one or more of the oligopeptides identified as SEQ ID No: 1 through SEQ ID NO: 118 and/or modifications thereof according to claims 2 to 10 and/or peptidomimetics according to claim 1 to 10 to indirectly reduce or block the metabolic action or interaction of African Swine Fever virus by applying them as vaccines by subcutaneous application or in another acceptable way to stimulate a specific immune response which can partially or completely block immune deficiencies.

18. The use of one or more of the oligopeptides identified as SEQ ID No: 1 through SEQ ID NO: 118 and/or modifications thereof according to claims 2 to 10 and/or peptidomimetics according to claim 1 to 129 to prevent or treat immune deficiencies in any other medically acceptable way.

19. The use of one or more of the oligopeptides identified as SEQ ID No: 1 through SEQ ID NO: 118 and/or modifications thereof according to claims 2 to 10 and/or peptidomimetics according to claim 1 to 10 for the prevention or therapy of infectious diseases.

20. The use of one or more of the oligopeptides identified as SEQ ID No: 1 through SEQ ID NO: 118 and/or modifications thereof according to claim 2 to 10 and/or peptidomimetics according to claim 1 to 10 for the prevention or therapy of diseases that may turn out to be caused or related to African Swine Fever virus.

21. The preventive and therapeutic use of one or more of the oligopeptides identified as SEQ ID No: 1 through SEQ ID NO: 118 and/or modifications thereof according to claim 2 to 10 and/or peptidomimetics according to claim 1 to 10 where the oligopeptides are coupled to haptens to enhance immune response and thereby therapeutic efficacy.

22. The use of one or more of the oligopeptides of claim 1-118 and/or modifications thereof identified as SEQ ID No: 1 through SEQ ID NO: 118 and/or peptidomimetics according to claim 1 to 10 for the production of specific antibodies for the diagnosis of a disease involving African Swine Fever virus or the clinical monitoring of the progression or regression of this disease.

23. The use of one or more of the oligopeptides identified as SEQ ID No: 1 through SEQ ID NO: 118 and/or modifications thereof according to claim 2 to 10 and/or peptidomimetics according to claim 1 to 10 for the production of specific antibodies for the diagnosis or the clinical monitoring of the progression or regression of immune deficiencies.

24. The use of one or more of SEQ ID NO: 1 to 32 and SEQ ID NO: 34 to 38 where these oligopeptides can be applied to a patient as a vaccine, as injections, infusions, inhalations, suppositories or other pharmaceutically acceptable carriers and/or means of delivery.
